(19) United States
(12) Patent Application Publication
(10) Pub. No.: US 2019/0126002 A1
(43) Pub. Date: May 2, 2019

(54) METHOD FOR USING TARGETED PLAYLISTS TO TREAT DEMENTIA PATIENTS

(71) Applicant: Tim Brimmer, Zionsville, IN (US)

(72) Inventor: Tim Brimmer, Zionsville, IN (US)

(21) Appl. No.: 15/962,204

(22) Filed: Apr. 25, 2018

Related U.S. Application Data
(63) Continuation-in-part of application No. 15/799,228, filed on Oct. 31, 2017.

(51) Int. Cl. A61M 21/02; G06F 3/16

(52) U.S. Cl. CPC A61M 21/02; G06F 3/16; A61M 2205/3303; A61M 2205/3375; A61M 2021/0027; A61M 2230/10; A61M 2205/3306; A61M 2230/63; A61M 2230/04

(57) ABSTRACT

A system for treating a patient with music, including an audio playback, a speaker operationally connected to the audio playback, a controller operationally connected to the audio playback, a plurality of respective personalized playlists operationally connected to the audio playback, and a sensor array operationally connected to the controller for providing a combination of sensor signals thereto. Each respective personalized playlist is associated with a respective predetermined patient state, and the controller is programmed to associate respective predetermined combinations of sensor signals with respective predetermined patient states. The controller actuates the audio playback to play music from a respective personalized playlist upon detection of the predetermined patient state associated with the respective personalized playlist.

### TECHNICAL FIELD

The present novel technology relates generally to acoustical engineering and, more particularly, to method of customizing audio playback devices to provide targeted treatment music to dementia patients.

### BACKGROUND

Some residents in Alzheimer's disease- and dementia-care facilities exhibit severe agitation and unprovoked loud, aggressive, and disruptive outbursts and behaviors, often generalized and without any specific target. At other times, these patients present with lethargy and listlessness, sadness, or the like. These behaviors often prove to be irritating, if not outright dangerous, to the patient and to others, highly disturbing to other residents, and can set off a ‘chain reaction’ of agitation, crying, or the like. Currently, such unwanted behaviors, escalations, or outbursts are almost universally treated by the pro re nata (PRN) application of drugs and medications, often carrying a black-boxed or black-label warning, signifying extreme health risks and hazards.

One obvious major drawback with this treatment strategy is that it is adverse to the patient's long-term health. Another is that once the patient starts down the slippery slope of powerful drugs, what remains left of the patient's mind and personality has little chance of reemergence, at least while the influence of the drug remains in effect. Moreover, the outbursts often resume as soon as the medication is discontinued. Thus, there remains a need for an improved patient care strategy to address escalating agitation or like emotional states in dementia patients. The present novel technology addresses this need.

### BRIEF DESCRIPTION OF THE DRAWINGS

FIG. 1 is a block diagram a first embodiment of a patient soothing system of the present novel technology.

FIG. 2 is a block diagram view of the first embodiment of the patient soothing system of claim 1 in use with a patient.

FIG. 3 a block diagram of an example environment in which the patient soothing system of claim 1 may exist.

FIG. 4 is an example system diagram of an example computer system that may be used to create the patient soothing device system of claim 1.

FIG. 5 is a process flow diagram associated with a first method associated with the patient soothing system of claim 1.

FIG. 6 is a process flow diagram associated with a second method associated with the patient soothing system of claim 1.

FIG. 7 is a block diagram a second embodiment of a patient soothing system of the present novel technology.

FIG. 8 is a process flow diagram associated with a first method associated with the patient soothing system of claim 7.

Like reference numbers and designations in the various drawings indicate like elements.

### DETAILED DESCRIPTION

For the purposes of promoting an understanding of the principles of the invention and presenting its currently understood best mode of operation, reference will now be made to the embodiments illustrated in the drawings. It will nevertheless be understood that no limitations of the scope of the invention is intended by the specific language used to describe the invention, with such alterations and further modifications in the illustrated device and such further applications of the principles of the invention as illustrated therein being contemplated as would normally occur to one ordinarily skilled in the art.

As used in the specification and the claims, the singular forms “a,” “an” and “the” include plural referents unless the context clearly dictates otherwise. Ranges may be expressed in ways including from “about” one particular value, and/or to “about” another particular value. When such a range is expressed, another implementation may include from the one particular value and/or to the other particular value. Similarly, when values are expressed as approximations, for example by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Similarly, “typical” or “typically” means that the subsequently described event or circumstance often though may not occur, and that the description includes instances where said event or circumstance occurs and instances where it does not. Additionally, “generates,” “populates,” “generating,” and “populating” mean that the system 10, user, and/or module may produce some event or cause some event element to be produced. For example, system 10 may receive data to display in whole or in part an example playlist 25 for review by caretakers, family members, or the patient during lucidity.

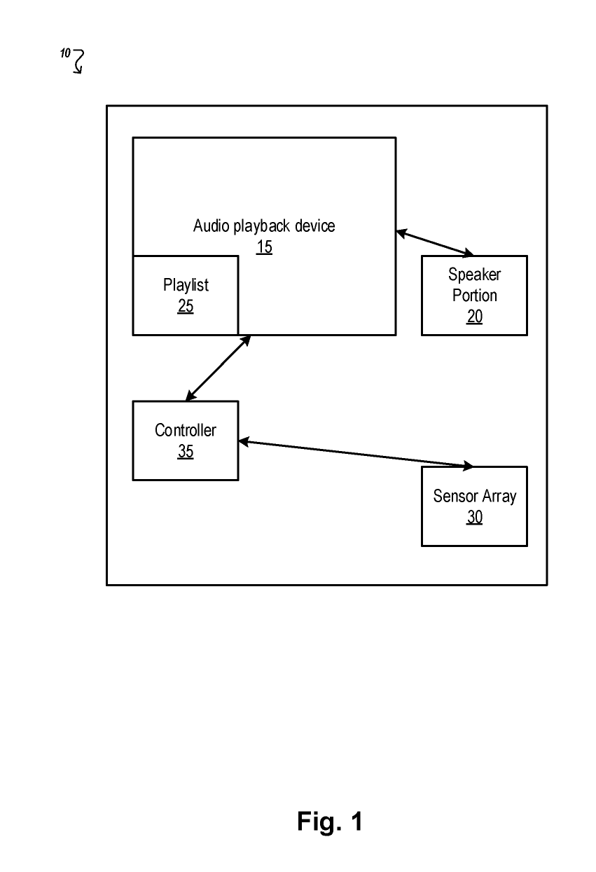

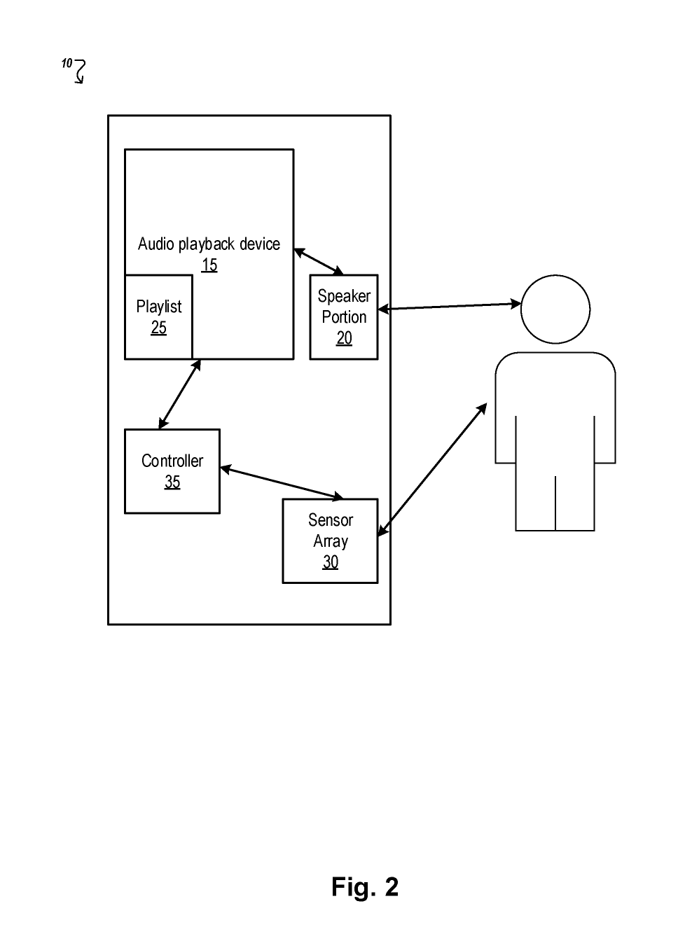

As illustrated in FIGS. 1-8, the present novel technology relates to a (typically automated) patient soothing system 10 for providing relief to a distressed patient, such as a patient suffering from Alzheimer's disease, senile dementia, or like afflictions or conditions, or anyone suffering from agitation. The system 10 includes an audio playback portion 15 and a speaker portion 20 operationally connected to the audio playback portion 15. The audio playback portion 15 is loaded with a personalized playlist 25, in this example an agitation-soothing playlist 25A. The playlist 25A is typically about thirty to sixty minutes long, although it can be any convenient length supported by the playback device 15. The playback device 15 is typically an IPOD SHUFFLE (registered trademark of Apple Inc., a California Corporation located at 1 Infinite Loop, Cupertino Calif. 95014, reg. no. 3453564), smart phone, and/or like personal audio device. Likewise, the speaker portion 20 is typically a set of over-the-ear (circumaural) headphones, although any like personal speaker system may be selected. For example, a patient's room may be set up with a speaker or speaker system (typically high-fidelity) connected to a local, remote, and/or like playback device 15.

Connectivity between devices (e.g., playback portion 15, speaker portion 20, playlist 25, sensor array 30, controller 35, and/or the like) and/or between system 10 and environment 300 may be wired (such as through 3.5 mm, aux, coaxial, RJ11/RJ45, fiber optic, etc. connections) and/or wireless, such as through BLUETOOTH (BLUETOOTH is a registered trademark of SIG, Inc., a Delaware corporation, located at 5209 Lake Washington Boulevard, Suite 350, Kirkland, Wash. 98033), APTX (APTX is a registered trademark of Qualcomm Technologies International, Ltd., a United Kingdom corporation. Located at Churchill House, Cambridge Business Park, Cowley Road, Cambridge, United Kingdom CB4 0WZ), AIRPLAY (AIRPLAY is a registered trademark of Apple Inc., a California corporation, located at 1 Infinity Loop, Cupertino, Calif. 95014), ZIGBEE (ZIGBEE is a registered trademark of ZigBee Alliance, a California corporation, located at 2400 Camino Ramon, Suite 375, San Ramon, Calif. 94583), ZWAVE (ZWAVE is a registered trademark of Sigma Designs, Inc., a California corporation, located at 47467 Fremont Blvd., Fremont, Calif. 94538), radio frequency, infrared, and/or other technologies. Some implementations may be purely wired, purely wireless, and/or a combination thereof.

For example, playback device 15 and speaker 20 may be connected via BLUETOOTH for playback and control of playlist 25 (in this example, an agitation-soothing playlist 25A), and the sensor array 30 (in this example, agitation sensor 30A) may connect to playback device 15 and/or controller 35 over an IEEE 802.11 wireless data link. In other implementations, playback device 15 and speaker 20 may be connected via AIRPLAY for playback and control of playlist 25A, agitation sensor 30A may connect to controller 35 over an Ethernet link, and controller 35 may communicate with playback device and speaker 20 over an IEEE 802.11 wireless data link. Further, in some implementations where playback device 15, speaker 20, and controller 35 are combined (e.g., as a smart speaker), agitation sensor 30A may communicate to the combined device to control playback of playlist 25A.

In general, the personalized playlist 25 is assembled using several criteria, such as patient's age or birthdate; the geographic residential history, specifically the geographic area occupied by the patient between the ages of about ten and about twenty-five (more typically between the ages of about fourteen and about twenty-two); the patient's musical favorites as told by patient, patient's family and/or friends; songs and/or artists found on patient's ITUNES (a registered trademark of Apple Inc., a California Corporation located at 1 Infinite Loop, Cupertino Calif. 95014, reg. no. 3452063), AMAZONMUSIC (a registered trademark of Amazon Technologies, Inc., a Nevada corporation located at 410 Terry Ave. N., ATTN: Trademarks, Seattle Wash. 98109, reg. no. 4918824), SPOTIFY (a registered trademark of Spotify AB Corporation of Birger Jarlsgatan 61 Stockholm SWEDEN 5E113 56, reg. no. 3561218), and/or like commercial playlist(s) (with emphasis on most routinely played or ‘favorite’ songs); patient's education and educational history; patient's occupation; patient's military service; patient's ethnicity; and/or like factors.

The playlist 25A, once assembled, may be edited (manually, semi-automatically, and/or automatically) based on directly imputed, observed, or otherwise collected feedback. For example, a caregiver may delete a song that caused patient distress when played, or may give extra weight to a song that has great quieting effect on the patient. The system 10 may include an agitation sensor 30A (motion, voice-activated, and/or the like) and/or electronic controller 35 operationally connected to the audio playback device 15 that initiates the device 15 to skip playback of a song that is associated with increasing agitation and/or override playback to initiate performance of a song weighted for its calming effect. In some implementations, electronic controller 35 may operate remotely as remote controller 35, which may more easily and quickly respond to the patient's current state of agitation. Such interactions may be stored for future reference and generation of playlists 25A (for example, in playlist datastore 360, described below).

The audio playback device 15, through attachment of the headphones 20, may be engaged to provide patient with soothing, personalized, and selected music from the playlist 25A at regular prescribed intervals, when needed (that is, according to PRN), such as when the patient becomes agitated (initiated manually and/or automatically), preventatively in anticipation of a disruptive event, and/or upon request of the patient, patient's family, or a caregiver.

In practice, personal information is gathered regarding the patient, such as age, exposure to popular music during key years (such as from fifteen to twenty-three years of age), genre preferences, favorite songs and tunes, and/or the like, and said information is used to populate a playlist 25A, the songs from which are then loaded into a playback device 15.

In operation, the system 10 is engaged to the patient, such as having the audio playback portion 15 clipped or fastened to the patient's chair or clothing, and the headphone portion 20 positioned over the patient's ears. The playback portion 15 is energized (either automatically or manually, such as by the caregiver or by the patient) to play music from the patient's playlist 25A to the patient. More highly functioning patients may be granted access to skip around their playlist 25A as desired. The playlist 25A is edited and updated, such as automatically by electronic controller 35, or manually by the caregiver or patient as music is played.

Electronic controller 35 may, for example, be a system independent of audio playback device 15, or a subpart of device 15. For example, where device 15 is a smartphone or the like, controller 35 may be integrated with device 15 to generate queries 330 based on patient data 350, provide queries 330 to third-party resources 320, receive results 340, store and query playlist data 360, and/or the like (described in greater detail below). Electronic controller 35 may also be a separate hardware module designed to provide system 10 features (e.g., demographic query generation, music transmission, etc.), for example where playback device 15 is more specifically tailored for media playback (e.g., IPOD, audio receiver, etc.).

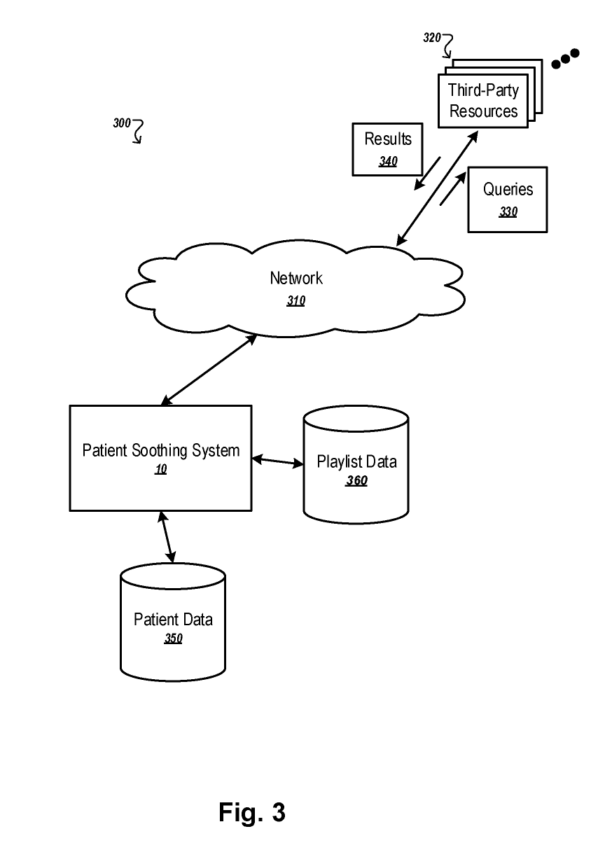

FIG. 3 is a block diagram of an example environment 300 in which patient soothing system 10 may exist. Environment 300 may typically include patient soothing system 10, network 310, third-party resources 320, queries 330, results 340, patient data 350, and playlist data 360. Example environment 300 includes network 310, such as a local area network (LAN), a wide area network (WAN), the Internet, and/or a combination thereof. Network 310 typically may connect third-party resources 320 and patient soothing system 10. Example environment 300 may potentially include many thousands of third-party resources 320 and/or patient soothing systems 10.

Third-party resources 320 may be one or more resources associated with a domain name and/or hosted by one or more servers. An example third-party resources 320 may be a collection of webpages formatted in hypertext markup language (HTML) that may contain text, images, multimedia content, and programming elements, such as scripts. Third-party resources 320 may also be a music subscription service, geographic data, ethnographic data, historical media data, social networks, and/or the like. Each third-party resources 320 may be maintained by a publisher, which may be an entity that controls, manages, and/or owns each third-party resource 320.

Resource(s) 320 may be any data that may be provided over network 310. Resource(s) 320 may be identified by a resource address (e.g., a URL) that may be associated with the resource(s) 320. Resources 320 include HTML webpages, word processing documents, and portable document format (PDF) documents, images, music files, video, and/or feed sources, to name only a few. Resources 320 may include content, such as words, phrases, images and sounds, that may include embedded information—such as meta-information in hyperlinks—and/or embedded instructions, such as JAVASCRIPT scripts (JAVASCRIPT is a registered trademark of Sun Microsystems, Inc., a Delaware corporation, located at 4150 Network Circle Santa Clara, Calif. 95054).

Patient soothing system 10 typically may facilitate querying 330 and receiving results 340 from third-party resources 320 via input (e.g., typing on touchscreen, etc.) on playback device 15. Playback devices 15 typically may be electronic devices that are under the control of an end user (e.g., caregiver, family member, etc.) and may be capable of requesting and receiving resources 320 over network 310 via queries 330 and results 340. Playback devices 15, in some implementations, typically may also include a user application, such as a web browser, to facilitate the sending and receiving of data over the network 310 and to/from resources 320.

In some implementations, third-party resources 320, playback devices 15, and systems 10 may directly intercommunicate, excluding the need for the Internet from the scope of a network 310. For example, third-party resources 320, playback device 15, and the patient soothing system 10 may directly communicate over device-to-device (D2D) communication protocols (e.g., BLUETOOTH, WI-FI DIRECT (WI-FI DIRECT is a registered trademark of Wi-Fi Alliance, a California corporation, located at 10900-B Stonelake Boulevard, Suite 126, Austin, Tex. 78759); Long Term Evolution (LTE) D2D (LTE is a registered trademark of Institut Europeen des Normes; a French nonprofit telecommunication association, located at 650 route des Lucioles, F-06921, Sophia Antipolis, France), LTE Advanced (LTE-A) D2D, etc.), wireless wide area networks, and/or satellite links thus eliminate the need for the network 310 entirely. In other implementations, third-party resources 320, playback devices 15, and system 10 may communicate indirectly to the exclusion of the Internet from the scope of the network 310 by communicating over wireless wide area networks and/or satellite links. Further, playback device 15 may similarly send and receive data queries 330 and data results 340 indirectly or directly.

In wireless wide area networks, communication primarily occurs through the transmission of radio signals over analog, digital cellular, or personal communications service (PCS) networks. Signals may also be transmitted through microwaves and other electromagnetic waves. At the present time, most wireless data communication takes place across cellular systems using second generation technology such as code-division multiple access (CDMA), time division multiple access (TDMA), the Global System for Mobile Communications (GSM) (GSM is a registered trademark of GSM MoU Association, a Swiss association, located at Third Floor Block 2, Deansgrande Business Park, Deansgrande, Co Dublin, Ireland), Third Generation (wideband or 3G), Fourth Generation (broadband or 4G), personal digital cellular (PDC), or through packet-data technology over analog systems such as cellular digital packet data (CDPD) used on the Advance Mobile Phone System (AMPS).

The patient soothing system 10 may use one or more software modules to perform various functions including, but not limited to, searching, analyzing, querying, interfacing, etc. A “module” refers to a portion of a computer system and/or software program that carries out one or more specific functions and may be used alone or combined with other modules of the same system or program. For example, a module may be located on the patient soothing system 10 (e.g., on a server associated with system 10, i.e., server-side module), on playback device 15, or on an intermediary device (e.g., the client server, i.e., a client-side module; another playback device 15; a different server on the network 310; or any other machine capable of direct or indirect communication with system 10, third-party resources 320, and/or the playback devices 15.)

In some implementations, generation of system queries 330 using patient data 350 (e.g., patient birthdate, home town, education, work background, etc.) and receipt of results 340 to create playlists 25 (typically stored on playback devices 15 and/or playlist data 360) may be performed through a module. For example, a caregiver may install a program to interface with a system 10 and patient datastore 350 to generate a relevant query 330 to third-party resources 320. For example, such queries 330 may request most played songs on a patient's SPOTIFY playlist; the highest charting songs in Denver, Colorado in 1952 (i.e., where Denver was the patient's home); and/or the most played songs from 1964-1968 (i.e., where patient was in college during this time period). Third-party resources 320 may then send results 340 back to system 10 and module, which then stores relevant information (songs, artists, length of time charting, patient's preferences) to playback device 15 and/or playlist datastore 360. In some implementations, system 10 may also query stored information in patient datastore and/or playlist datastore 360 to augment playback and playlists 25. For example, where a particular song has a great calming effect, that song may be permanently cached and typically imported into playlists, or, conversely, where a song is a hit but agitates patient, it may be permanently flagged for removal from playlists 25.

In some other implementations, the system module may be installed on a server to operate—in whole or in part—independently of system 10. For example, the system module may be deployed to a caregiving facility's computer as a standalone program that interfaces with the third-party resources 320, creates and maintains data store(s), generates playlists 25, parses stored preferences, transmits playlists 25 to playback devices 15, etc.

Typically, modules may be coded in JAVASCRIPT, PHP, or HTML, but may be created using any known programming language (e.g., BASIC, FORTRAN, C, C++, C#, PERL (PERL is a registered trademark of Yet Another Society DBA The Perl Foundation, a Michigan nonprofit corporation, located at 340 S. Lemon Ave. #6055, Walnut, Calif. 91789)) and/or package (e.g., compressed file (e.g., zip, gzip, 7zip, RAR (RAR is a registered trademark of Alexander Roshal, an individual, located in the Russian Federation AlgoComp Ltd., Kosareva 52b-83, Chelyabinsk, Russian Federation 454106), etc.), executable, etc.).

In other implementations, patient soothing system 10 software may be installed in whole or in part on an intermediary system that may be separate from the caregiver and system 10. For example, patient soothing system 10 software may be installed onto a hosting service (e.g., AMAZON WEB SERVICES (AWS) (AWS is a registered trademark of Amazon Technologies, Inc., a Nevada corporation, located at PO Box 8102, Reno, Nev. 89507), RACKSPACE (RACKSPACE is a registered trademark of Rackspace US, Inc., a Delaware corporation, located at 1 Fanatical Place, City of Windcrest, San Antonio, Tex. 78218), etc. The caregiver may then connect to the intermediary servers to access and/or generate playlists 25. Such implementations may, for example, allow distributed access, redundancy, decreased latency, etc.

In some implementations, user interaction data may be anonymized to protect the identity of the patient with which the user interaction data is associated. For example, when making queries, user identifiers may be removed from the user interaction data. Alternatively, the user interaction data may be associated with a hash value of the user identifier to anonymize the user identifier. In some implementations, user interaction data are only stored for users that opt-in to having user interaction data stored. For example, a user may be provided an opt-in/opt-out user interface that allows the user to specify whether they approve storage of data representing their interactions with content. Such privacy protections may be useful for providing patient care without running into regulatory restrictions of protected health information or personally identifiable information.

In some implementations, the interaction apparatus 170 may also determine that the portion of all users that performed a predictive interaction, but did not perform the target interaction. The interaction apparatus 170 may use this determination as an indication of the false positive rate that may occur using the predictive interaction as a proxy for the target interaction.

Once the interaction apparatus 170 selects the predictive interactions, the interaction apparatus 170 determines whether additional user interaction data include predictive interaction data. The additional user interaction data may be user interaction data that do not include target interaction data. For example, the additional user interaction data may be user interaction data for user interactions with a website for which click-throughs are not tracked. When the interaction apparatus 170 determines that the additional user interaction data include the predictive interaction data, the user from which the user interaction data was received may be considered a click-through user for purposes of determining content item effectiveness.

In some implementations, the interaction apparatus 170 may assign each click-through user a weight that represents the relative importance of the click-through user's interactions for computing content item effectiveness. For example, a user that performs many different predictive interactions may have a higher weight than a user that performs only one predictive interaction. In some implementations, the interaction apparatus 170 may assign a same weight—that is, 1.0—to each click-through user. This concept may be used to more accurately correlate and suggest content to users. For example, if a user typically interacts with results corresponding to new entity or service proposals, then the system 10 may weight results of new entity or services above older entities. Additionally, the system 10 may give greater weight to a user that more closely correlates to another user. For example, if one user typically interacts or searches for software companies in a similar fashion to the way in which another user typically interacts or searches, then the searches or interactions of one user may be suggested to the other in certain circumstances. Other correlation methods may also be used, such as cosine similarity measures, clustering techniques, or any other similar technique.

Further, in some implementations, the system 10 may utilize machine learning and/or weighting algorithms to better determine similarity weightings between media, patients, and/or demographics, a weighting being a value representing an objective similarity between a first sample and a second sample based on a multitude of factors including, but not limited to, number of shared demographic categories, frequency of agitation/calming, intensity of agitation/calming, etc. For example, if Patient A shares five demographic classifiers (e.g., age, state, military service, education, family size, political party, etc.) in common with Patient B, but shares ten interests in common with User C, then User A may be assigned a higher similarity weight with User C than with User B. In some implementations, the factors affecting the similarity weight may be given equal weight, while in other implementations the weight given to each factor may vary based on some subjective or objective weighing scheme.

In some implementations, suggestions may be given to a playlist 25 based on the similarity weight, among many other possible factors. In other implementations, similarity weightings may be given between patients based on response to various songs or playlists 25. For example, where Patient D and Patient E only have two shared classifiers, but have a highly correlative response to a specific album, Patient D may be suggested other media that is effective with Patient E that otherwise might not be suggested based solely on demographic classifiers. This may, for example, be due to a shared life experience (e.g., first dance with spouses) or any number of other edge-case factors. As such, machine learning may be applied to develop unique profiles and correlations between patients and music that may otherwise be deemed irrelevant by raw personal demographics, substantially increasing the effectiveness of system 10 and playlist 25 generation for treatment.

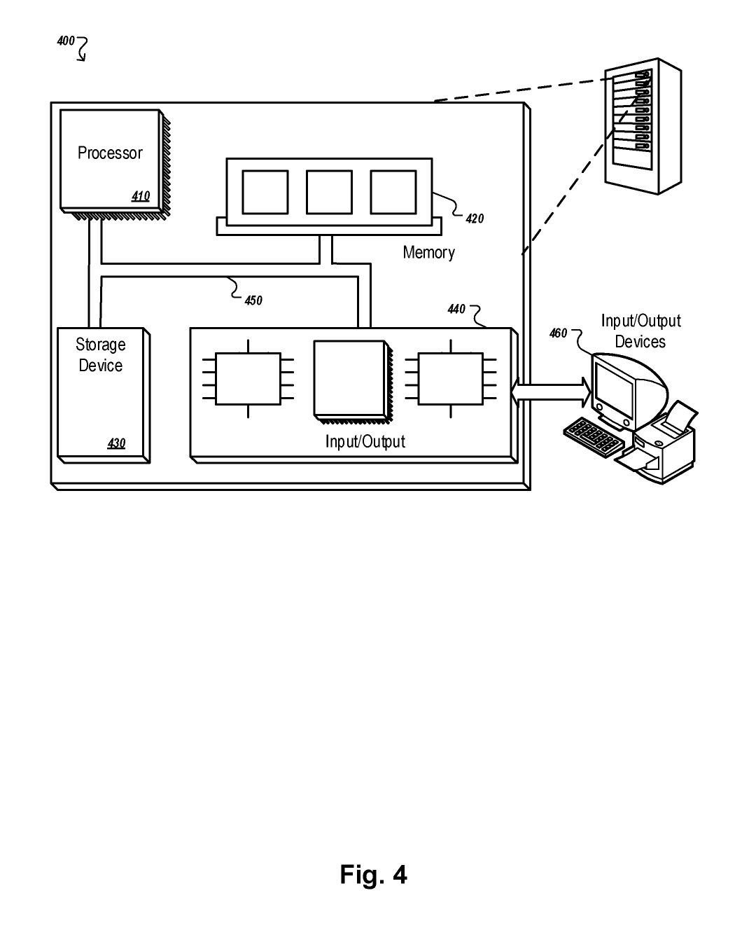

FIG. 4 is a block diagram of an example computer system 400 that may be used to provide patient soothing system 10, as described above. System 400 may, for example, exist on playback device 15 and/or another server that interfaces with device 15. The computer system 400 may typically include processor(s) 410; memory 420; storage device(s) 430; system input(s)/output(s) 440; system bus(es) 450; and input/output device(s) 460. Each of the components 410, 420, 430, and 440 typically may be interconnected, for example, using system bus(es) 450. Processor(s) 410 may be capable of processing instructions for execution within the system 00. In one implementation, processor(s) 410 may be a single-threaded processor. In another implementation, processor(s) 410 may be a multi-threaded processor. In yet another implementation, processor(s) 410 may be a single-core processor, a multiple-core processor, and/or multiple processors (i.e., more than one socketed processor). Processor(s) 410 typically may be capable of processing instructions stored in the memory 420 and/or on the storage device(s) 430.

Memory 420 stores information within system 400. In one implementation, memory 420 may be a computer-readable medium. In one other implementation, memory 420 may be a volatile memory unit. In another implementation, memory 420 may be a nonvolatile memory unit.

Storage device(s) 430 may be capable of providing mass storage for the system 400. In one implementation, storage device(s) 430 may be a computer-readable medium. In various different implementations, storage device(s) 430 may include, for example, a hard disk device, a solid-state disk device, an optical disk device, and/or some other large capacity storage device. For example, such storage device 430 may contain playlist datastore 360 and/or patient database 350.

System input(s)/output(s) 440 provide input/output operations for the system 400. In one implementation, system input(s)/output(s) 440 may include one or more of a network interface devices, for example an Ethernet card; a serial communication device, for example an RS-232 port; and/or a wireless interface device (e.g., BLUETOOTH, IEEE 802.11 card, etc.). In another implementation, system input(s)/output(s) 440 may include driver devices configured to receive input data and send output data to other input/output device(s) 460, for example keyboards, printers, display devices, and/or any other input/output device(s) 460. Other implementations, however, may also be used, such as mobile computing devices, mobile communication devices, set-top box television client devices, etc.

Although an example processing system has been described in FIG. 4, implementations of the subject matter and the functional operations described in this specification may be implemented in other types of digital electronic circuitry, or in computer software, firmware, or hardware, including the structures disclosed in this specification and their structural equivalents, or in combinations of one or more of them.

Embodiments of the subject matter and the operations described in this specification may be implemented as a method, in digital electronic circuitry, or in computer software, firmware, or hardware, including the structures disclosed in this specification and their structural equivalents, or in combinations of one or more of them. Embodiments of the subject matter described in this specification may be implemented as one or more computer programs—that is, one or more modules of computer program instructions encoded on computer storage medium for execution by, or to control the operation of, data processing apparatus. Alternatively or in addition, the program instructions may be encoded on an artificially generated propagated signal, for example a machine-generated electrical, optical, or electromagnetic signal, which may be generated to encode information for transmission to suitable receiver apparatus for execution by a data processing apparatus. A computer storage medium may be, or be included in, a computer-readable storage device, a computer-readable storage substrate, a random or serial access memory array or device, or a combination of one or more of them. Moreover, while a computer storage medium may not be a propagated signal, a computer storage medium may be a source or destination of computer program instructions encoded in an artificially generated propagated signal. The computer storage medium may also be, or be included in, one or more separate physical components or media (e.g., multiple CDs, disks, or other storage devices).

The operations described in this specification may be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus may include special purpose logic circuitry, for example an field programmable gate array (FPGA) or an application specific integrated circuit (ASIC). The apparatus may also include, in addition to hardware, code that creates an execution environment for the computer program in question, for example code that constitutes processor firmware, a protocol stack, a database management system, an operating system, a cross-platform runtime environment, a virtual machine, or a combination of one or more of them. The apparatus and execution environment may realize various different computing model infrastructures, such as web services, distributed computing, and grid computing infrastructures.

A computer program (also known as a program, software, software application, script, or code) may be written in any form of programming language, including compiled or interpreted languages, declarative or procedural languages, and it may be deployed in any form, including as a standalone program or as a module, component, subroutine, object, or other unit suitable for use in a computing environment. A computer program can, but need not, correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one computer or on multiple computers that are located at one site or distributed across multiple sites and interconnected by a communication network.

The processes and logic flows described in this specification may be performed by one or more programmable processors executing one or more computer programs to perform actions by operating on input data and generating output. The processes and logic flows may also be performed by, and apparatus may also be implemented as, special purpose logic circuitry, for example an FPGA or an ASIC.

Processors suitable for the execution of a computer program include, by way of example, both general and special purpose microprocessors, and any one or more processors of any kind of digital computer. Typically, a processor may receive instructions and data from a read only memory or a random access memory or both. The essential elements of a computer are a processor for performing actions in accordance with instructions and one or more memory devices for storing instructions and data. Typically, a computer may also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, for example a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, for example erasable programmable read-only memory (EPROM), electrically erasable programmable read-only memory (EEPROM), and/or flash memory devices; magnetic disks, for example internal hard disks or removable disks; magneto optical disks; and/or compact disk read-only memory (CD ROM) and digital video disk real-only memory (DVD-ROM) disks. The processor and the memory may be supplemented by, or incorporated in, special purpose logic circuitry.

To provide for interaction with a user, embodiments of the subject matter described in this specification may be implemented on a device having a display (e.g., a cathode ray tube (CRT), liquid crystal display (LCD), or organic light-emitting diode (OLED) monitor), for displaying information to the user and a keyboard and a pointing device, e.g., a mouse or a trackball, by which the user may provide input to the computer. These may, for example, be desktop computers, laptop computers, smart TVs, etc. Other mechanisms of input may include portable and or console entertainment systems such as GAME BOY and/or NINTENDO DS (GAME BOY, GAME BOY COLOR, GAME BOY ADVANCE, NINTENDO DS, NINTENDO 2DS, and NINTENDO 3DS are registered trademarks of Nintendo of America Inc., a Washington corporation, located at 4600150th Avenue NE, Redmond, Wash. 98052), IPOD (IPOD is a registered trademark of Apple Inc., a California corporation, located at 1 Infinite Loop, Cupertino, Calif. 95014), XBOX (e.g., XBOX, XBOX ONE) (XBOX and XBOX ONE are a registered trademarks of Microsoft, a Washington corporation, located at One Microsoft Way, Redmond, Wash. 98052), PLAYSTATION (e.g., PLAYSTATION, PLAYSTATION 2, PS3, PS4, PLAYSTATION VITA) (PLAYSTATION, PLAYSTATION 2, PS3, PS4, and PLAYSTATION VITA are registered trademarks of Kabushiki Kaisha Sony Computer Entertainment TA, Sony Computer Entertainment Inc., a Japanese corporation, located at 1-7-1 Konan Minato-ku, Tokyo, 108-0075, Japan), OUYA (OUYA is a registered trademark of Ouya Inc., a Delaware corporation, located at 12243 Shetland Lane, Los Angeles, Calif. 90949), WII (e.g., WII, WII U) (WII and WII U are registered trademarks of Nintendo of America Inc., a Washington corporation, located at 4600 150th Avenue NE, Redmond, Wash. 98052), etc.

Other kinds of devices may be used to provide for interaction with a user as well; for example, feedback provided to the user may be any form of sensory feedback, for example visual feedback, auditory feedback, or tactile feedback; and input from the user may be received in any form, including acoustic, speech, or tactile input. In addition, a computer may interact with a user (e.g., patient, caregiver, etc.) by sending data to and receiving data from a device that may be used by the user; for example, by sending playlists 25 to a user's playback device 15 in response to requests received from the device 15.

Some embodiments of the subject matter described in this specification may be implemented in a computing system 400 that includes a back end component (e.g., a data server,) or that includes a middleware component (e.g., an application server,) or that includes a front-end component (e.g., a smartphone playback device 15 having a graphical user interface or a media player through which a user may interact with an implementation of the subject matter described in this specification,) or any combination of one or more such back end, middleware, or front-end components. The components of the computing system 400 may be interconnected by any form or medium of digital data communication, for example a communication network. Examples of communication networks include a local area network (LAN) and a wide area network (WAN), an inter-network (e.g., the Internet), and peer-to-peer networks (e.g., ad-hoc peer-to-peer, direct peer-to-peer, decentralized peer-to-peer, centralized peer-to-peer, etc.).

By way of nonlimiting examples, patient soothing system 10 may be constructed having audio playback portion 15 (e.g., an MP3 player), speaker portion 20, and personalized playlist 25 operationally connected to audio playback portion. The playlist 25 is personalized with music that was popular in the specific patient's location(s) when the patient was between the ages of fourteen and twenty-three years old. When the audio playback portion 15 is energized, it queries for a personalized playlist 25 and then plays the music from that playlist 25 to a patient through the speaker portion 20 (e.g., circumaural/ear covering headphones). As the agitated patient hears the personalized music playlist 25 that was relevant to him or her, the agitated patient typically may be calmed in an efficient and effective manner.

In some other implementations, an electronic controller 35 may be used to trigger the audio playback portion 15, and in further implementations, the electronic controller 35 may allow remote actuation to control playback portion 15. In still further implementations, system 10 may also include one or more agitation sensors 30 connected to the playback portion 15 and/or controller 35 to automatically energize and begin playback of playlist 25 upon receipt of a signal from the agitation sensor 30A. In some additional implementations, the playback portion 15 may be attachable to the patient's clothing or furniture. In still further implementations, electronic controller 35 may automatically update playback device 15 with newly identified music relevant to the agitated patient for one or more specified identifiers (e.g., age, location, education, etc.).

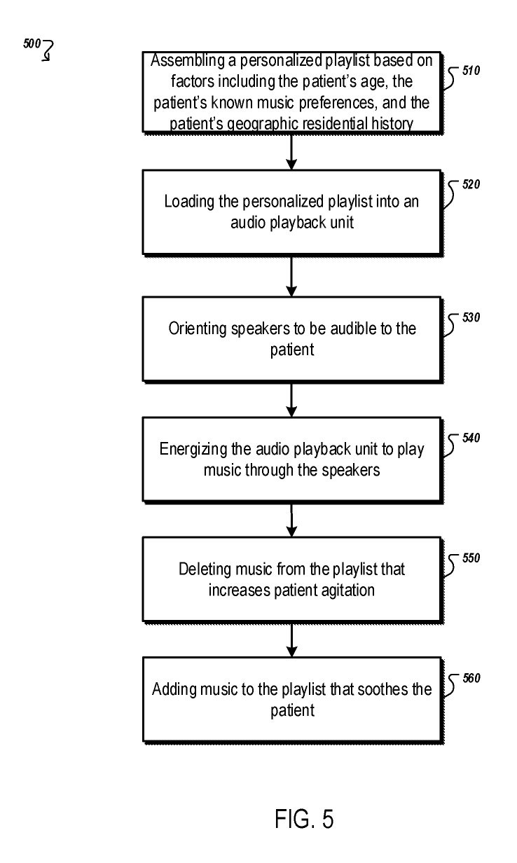

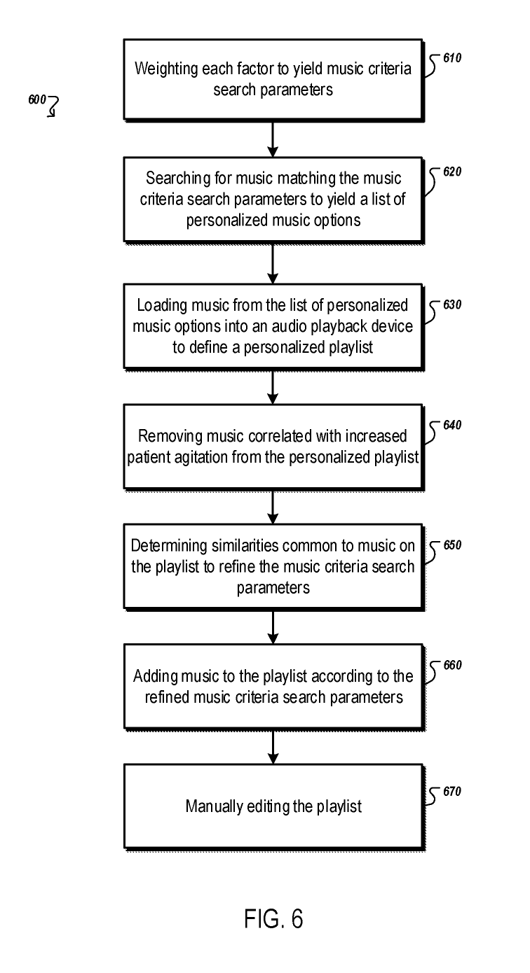

As depicted in FIGS. 5-6, patient soothing system 10 may also be used with a method for soothing an agitated dementia patient with music 500, the method 500 including the steps of assembling a personalized playlist based on factors including the patient's age, the patient's known music preferences, and the patient's geographic residential history 510; loading the personalized playlist into an audio playback unit 520; orienting speakers to be audible to the patient 530; energizing the audio playback unit to play music through the speakers 540; deleting music from the playlist that increases patient agitation 550; and adding music to the playlist that soothes the patient 560. Some other implementations of the method 500 including where the speakers are ear-covering headphones, where step 540 is performed automatically in response to patient agitation, and/or where the audio playback unit is operationally connected to an electronic controller for playlist updates and remote activation.

Another example method 600 utilizing system 10 is a method of producing a personalized playlist for a dementia patient 600, the method 600 including the steps of weighting each factor to yield music criteria search parameters 610; searching for music matching the music criteria search parameters to yield a list of personalized music options 620; loading music from the list of personalized music options into an audio playback device to define a personalized playlist 630; removing music correlated with increased patient agitation from the personalized playlist 640; determining similarities common to music on the playlist to refine the music criteria search parameters 650; and adding music to the playlist according to the refined music criteria search parameters 660. Further implementations may also include manually editing the playlist 670 and/or be configured where the audio playback unit is operationally connected to an electronic controller 35 for playlist updates and remote activation.

In such method example 600, the above-described similarity weighting algorithms and machine learning may be implemented to produce the personalized playlist 25. For example, such algorithm may take inputs from the patient datastore 350 to yield the music criteria search parameters as a query 330 to third-party resources and receive and search through results 340 of music matching music criteria search parameters to yield a list of personalized music options. The playlist 25 may then be loaded into the playback device 15 as a personalized playlist 25. For example, in the event that music is correlated with increased patient agitation from the personalized playlist 25A (e.g., from agitation sensor signal 30, controller 35, manual input, etc.), the music may be removed from the personalized playlist 25A. System 10 and/or controller 35 may also be used in determining similarities common to music on the playlist 25 to further refine music criteria search parameters for further queries 330, which may then be used to iteratively add music based on the refined queries 330. Further, in some implementations, the playlist 25 may be manually edited to add or remove music (e.g., music associated with known traumatic event and should not be played, music associated with first dance at wedding and should frequently be played, etc.), which may then be used to further refine queries 330 and playlist 25.

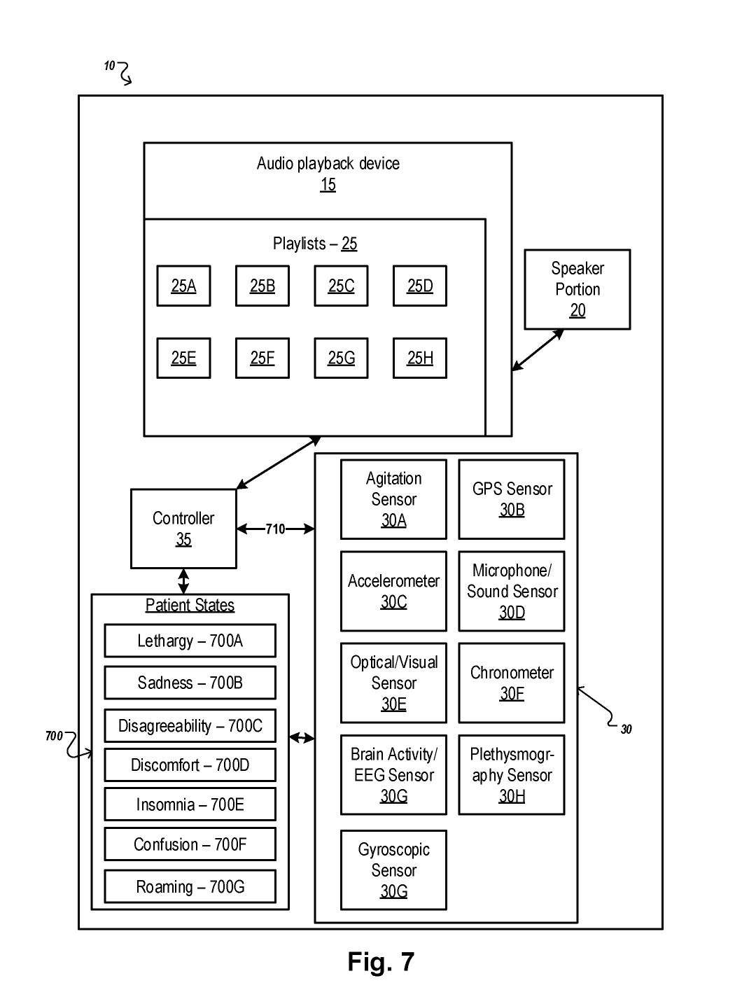

In another embodiment, typically depicted in FIG. 7, the system 10 may be expanded to detect and/or address other patient states 700 in addition to agitation, such as lethargy 700A, sadness 700B, disagreeability 700C, discomfort 700D, insomnia 700E, confusion 700F, roaming 700G, and the like. For example, dementia patients often present with sundowning syndrome, which may include one or more of the following symptoms: restlessness, disengagement, agitation, aggression, repetitive movements, unresponsiveness, and confusion. Detection of specific behaviors associated with specific symptoms, or combinations thereof, allows for treatment with an appropriate playlist tailored to elicit a specific response.

The sensor assembly 30 may include one or more specific detection devices such as an agitation sensor 30A, GPS sensor 30B, accelerometer 30C, microphone/sound sensor 30D (typically capable of measuring and differentiating changes in sound intensity, loudness, and sound pitch), optical/visual sensor 30E, chronometer 30F, brain activity/EEG sensor 30G, plethysmography sensor 30H, gyroscopic sensor 30I, pulse sensor 30J, blood pressure sensor 30K, and/or the like.

The sensor array 30 is operationally connected to the controller 35, which may be programmed to recognize certain combined sensor input signals, combinations, and/or patterns for correlation to specific physical and/or mental states, such as those recited above. Typically, the controller 35 would be capable of voice recognition, and more typically would be capable of identifying repetition or patterns of words or phrases as well as the utterance of specific predetermined words or phrases, associated with certain predetermined states, and would likewise be programmed to initiate playlists 25 paired with those identified words and phrases and patterns of the same.

Machine learning algorithms, as described above, and/or manual feedback may be employed to better correlate combinations of sensor signals 710 to a specific patient state 700 that, once identified, may be paired with specifically assembled playlists 25, as well as to automatically edit and reefing the respective playlists 25 to delete music that is correlated with negative efficacy (i.e., music that increases undesired behaviors or that fails to increase desired behaviors) and add music that is identified as likely to have the desired effect on behavior. Likewise, the controller 35 may correlate those songs most effective in treating the patient and assign so-identified songs high priority and/or frequent repeat appearances in a respective playlist 25. Such playlists could include an agitation soothing playlist 25A, an energizing or waking playlist 25B, a mood elevating or ‘happy’ playlist 25C, an agreeability encouraging playlist 25D, a comforting, reassuring playlist 25E, a bedtime, slumber-inducing playlist 25F, an attention-enhancing, concentration enabling playlist 25G, an attention-getting, stillness-inducing playlist 25H, or the like. Likewise, the controller 35 will automatically terminate playback (such as by turning the playback unit 15 off, or by slowly reducing the volume of the playback unit 15 until it reaches zero) when the combined sensor signals indicate that the patient is asleep. Playback from the energization/wake-up list 25B may be initiated at a predetermined time as determined by the chronometer sensor 30F.

The device 10 may include one or more detectors 30A-K of the sensor assembly 30 integral with the controller 35 and the playback 15 and speaker 20 hardware in a single, typically wearable, unit, such as a smart watch, ankle band, head band, belt, or the like. Alternately, the sensor assembly 30 and/or the speakers 20 may be physically remote from the controller and playback unit 15, which likewise may be integral or separate.

In this embodiment, device 10 may be able to use combined sensory inputs 710 to detect and identify specific behaviors associated with aggression, such as spitting, hitting, kicking, pushing, throwing objects, biting, scratching and the like, and initiate playback 15 of the appropriate list or lists 25. Likewise, the device 10 may be able to detect aberrant and/or repetitive movements, such as pacing, wandering, performance of repetitive mannerisms, tearing, general restlessness, exit seeking, roaming, and the like, and initiate playback 15 of the appropriate list or lists 25. The device 10 may also be able to detect specific predetermined verbal content and/or patterns associated with specific patient states, and initiate playback 15 of the appropriate list or lists 25.

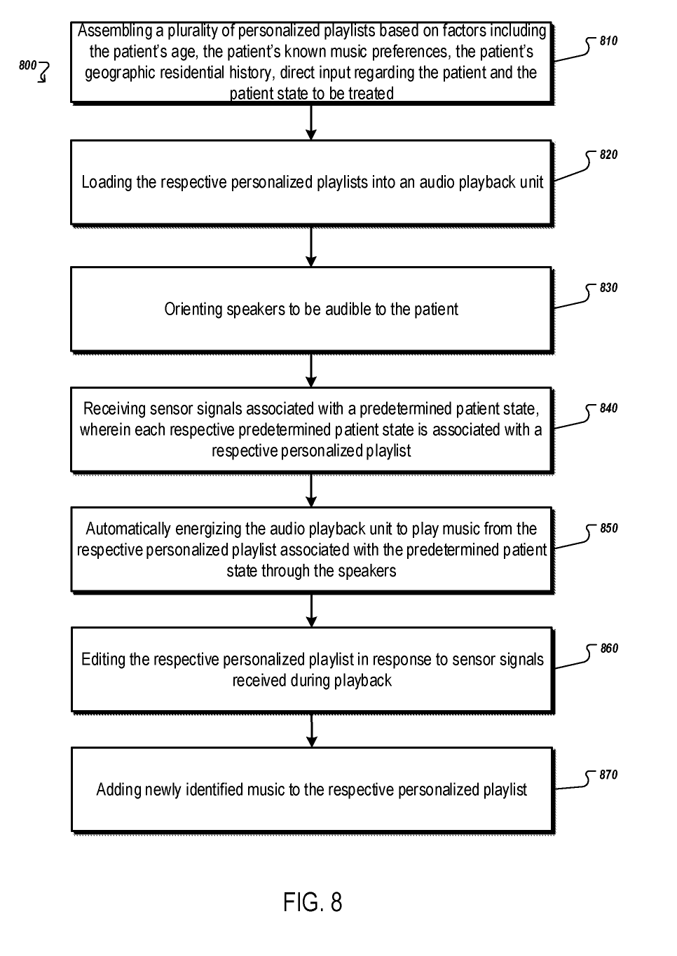

In a further implementation, typically depicted in FIG. 8, system 10 may be used in a method for treating a patient with music Boo, typically including the steps of “assembling a plurality of personalized playlists based on factors including the patient's age, the patient's known music preferences, the patient's geographic residential history, direct input regarding the patient and the patient state to be treated” 810; “loading the respective personalized playlists into an audio playback unit” 820; “orienting speakers to be audible to the patient” 830; “receiving sensor signals associated with a predetermined patient state, wherein each respective predetermined patient state is associated with a respective personalized playlist” 840; “automatically energizing the audio playback unit to play music from the respective personalized playlist associated with the predetermined patient state through the speakers” 850; “editing the respective personalized playlist in response to sensor signals received during playback” 860; and “adding newly identified music to the respective personalized playlist” 870.

In one specific example, as illustrated below in Table 1, a playlist is initially populated with music popular in the region where the patient lived while mid-teen through early 20's (such as during ages 14-23). The list is then modified by adding songs (from within or without the above age/geographic limitations), based on observed patient reactions while listening to music.

While the novel technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the novel technology are desired to be protected.

I claim:

1. A system for treating a patient with music, comprising:
an audio playback portion;
a speaker portion operationally connected to the audio playback portion;
a controller portion operationally connected to the audio playback portion;
a plurality of respective personalized playlists operationally connected to the audio playback portion; and
a sensor array portion operationally connected to the controller portion for providing a combination of sensor signals to the controller portion;
wherein each respective personalized playlist is associated with a respective predetermined patient state;
wherein the controller portion is programmed to associate respective predetermined combinations of sensor signals with respective predetermined patient states;
wherein the controller actuates the audio playback portion to play music from a respective personalized playlist upon detection of the predetermined patient state associated with the respective personalized playlist.

2. The system of claim 1 wherein the personalized playlist includes music popular where the agitated patient was located when the agitated patient was between 14 years old and 23 years old.

3. The system of claim 1 wherein the predetermined patient states are selected from the group comprising: lethargy, sadness, disagreeability, discomfort, insomnia, confusion, and roaming.

4. The system of claim 3 wherein the personalized playlists are selected from the group comprising: agitation soothing playlist, energizing playlist, mood elevating playlist, agreeability encouraging playlist, comforting playlist, slumber-inducing playlist, attention-enhancing playlist, and stillness-inducing playlist.

5. The system of claim 1 wherein each respective personalized playlist is automatically editable in response to patient feedback during playback to delete music correlated negative efficacy and to insert music identified as likely to have a desired effect on the predetermined patient state.

6. The system of claim 1 wherein the sensor array includes sensors selected from the group comprising: agitation sensors, GPS sensors, accelerometers, microphone/sound sensors, optical/visual sensors, chronometers, brain activity/EEG sensors, plethysmography sensors, gyroscopic sensors, and combinations thereof.

7. The system of claim 1 wherein the controller edits the playlist to assign a song higher priority in response to signals from the sensor array.

8. The system of claim 4 wherein the personalized playlist is automatically editable in response to signals from the sensor array during playback.

9. The system of claim 1 wherein the audio playback portion is attachable to the patient's clothing and wherein the speaker portion is a pair of ear-covering headphones.

10. The system of claim 2 wherein the electronic controller automatically updates the respective personalized playlists to include newly identified music popular where the patient was located when the patient was between 14 and 23 years old.

11. A method for treating a patient with music, comprising:
a) assembling a plurality of personalized playlists based on factors including the patient's age, the patient's known music preferences, the patient's geographic residential history, direct input regarding the patient and the patient state to be treated;
b) loading the respective personalized playlists into an audio playback unit;
c) orienting speakers to be audible to the patient;
d) receiving sensor signals associated with a predetermined patient state, wherein each respective predetermined patient state is associated with a respective personalized playlist;
e) automatically energizing the audio playback unit to play music from the respective personalized playlist associated with the predetermined patient state through the speakers;
f) editing the respective personalized playlist in response to sensor signals received during playback; and
g) adding newly identified music to the respective personalized playlist.

12. The method of claim ii, wherein the audio playback unit is operationally connected to an electronic controller for playlist updates and remote activation.

13. The system of claim ii wherein each respective personalized playlist is automatically editable in response to patient feedback during playback to delete music correlated negative efficacy and to insert music identified as likely to have a desired effect on the predetermined patient state; wherein the predetermined patient states are selected from the group comprising: lethargy, sadness, disagreeability, discomfort, insomnia, confusion, and roaming; and wherein the sensor array includes sensors selected from the group comprising: agitation sensors, GPS sensors, accelerometers, microphone/sound sensors, optical/visual sensors, chronometers, brain activity/EEG sensors, plethysmography sensors, gyroscopic sensors, and combinations thereof.
